(19) United States
(12) Patent Application Publication
(10) Pub. No.: US 2022/0221898 A1
(43) Pub. Date: Jul. 14, 2022

(54) TRACKING AND VALIDATING TIME OF PORTABLE DIGITAL RECORDING DEVICES

(71) Applicant: SIGNANT HEALTH OY, Helsinki (FI)

(72) Inventor: Sauli KAUPPI, Helsinki (FI)

(73) Assignee: SIGNANT HEALTH GLOBAL LLC, Blue Bell, PA (US)

(21) Appl. No.: 17/608,374

(22) PCT Filed: May 2, 2019
(86) PCT No.: PCT/FI2019/050345

§ 371 Date: Nov. 2, 2021

(51) Int. Cl. G06F 1/14; G07C 1/02

(52) U.S. Cl. CPC G06F 1/14; G07C 1/02

(57) ABSTRACT

An automatic time tracking and validating method of a portable digital device, including: maintaining by the portable digital device a current clock time and current uptime; and recording by the portable digital device a plurality of times a first entry that has: user information; the current clock time; the current uptime. A reference time recording process is performed once or more, including: recording by the portable digital device together with a server, which server has a reference time, a second entry that has: the reference time; the current clock time; and the current uptime. The portable digital device provides the server with the first and second entries.

### TECHNICAL FIELD

The present invention generally relates to tracking and validating time of portable digital recording devices. The invention relates particularly, though not exclusively, to tracking and validating time of portable digital clinical trial recording devices.

### BACKGROUND ART

This section illustrates useful background information without admission of any technique described herein representative of the state of the art.

Portable digital devices typically have clock circuitries that enables displaying current time and date as well as serving various applications and services run by these devices. Such clocks are typically freely adjustable by their users. Moreover, on running out of battery, such clocks may stop or even change to some initial default time. Still further, the clocks are often implemented using local oscillators the exact speed of which may depend on temperature, for example, so that some drifting (advancing or lagging behind) occurs.

Some portable digital devices can adapt to changes of time zone and to daylight saving such that the clock value may abruptly jump by one or more hours (e.g., after a long-haul flight).

Portable digital devices can be used for capturing data, such as clinical trial entries. Recorded entries may be automatically transmitted from the devices to a server with time stamps set based on the local clocks. Unless the recorded entries are continually sent immediately after their recording, the server has to rely on the time stamps provided by the devices.

Typically, in clinical trials, the digital devices are delivered to the clinical trial site for example by a party that collects and stores the captured data. In some cases, however, clinical trials could be made around a bring-your-own-device (BYOD) modality, wherein participants in a clinical trial use their own internet-enabled devices to provide study data.

Unless the digital devices have been set up to run in kiosk mode it may be impossible to prevent users from modifying the device's time and date. This is especially true with BYOD trials, wherein the party responsible for collecting the clinical trial data or setting up the digital devices for the trial or providing a dedicated application to be used in the trial may not be given rights to access the device's clock (by the user). Moreover, it might even be not permissible to control the device's clock by a party organizing the clinical trial. Yet, it is important to record observations of the user right on time. The users may not always remember or bother to punctually record notes, such as time of taking medication and generally answers to predefined questions or free text. If they can freely alter the time and date of their devices, it may be possible to falsely record observations as if they were timely entered.

It is desirable to track and validate the time of the devices.

### SUMMARY

According to a first example aspect of the invention there is provided an automatic time tracking and validating method of a portable digital device, comprising:
maintaining by the portable digital device a current clock time and current uptime;
recording by the portable digital device a plurality of times a first entry comprising:
   user information;
   the current clock time;
   the current uptime;
performing one or more times a reference time recording process comprising:
   recording by the portable digital device together with a server, which server has a reference time, a second entry that comprises: the reference time; the current clock time; and the current uptime; and
providing by the portable digital device the server with the first and second entries.

The method may further comprise using encryption by the portable digital device such that access of persons to the first and second entries is inhibited and integrity of the first and second entries is protected.

The automatic time tracking and validating method may enable tracking and validating times recorded for user information without exposing potentially sensitive information, e.g. to human beings. The automatic time tracking and validating method may enable tracking and validating times recorded for user information without necessarily having continuous online access.

The recording of the first entry may be performed in response to the portable digital device receiving from a user the user information.

The user information may comprise information provided by the user. The user information may comprise information describing the physical state of the user. The user information may comprise information describing the mental state of the user. The user information may be measured from the user. The user information may comprise clinical outcomes. The clinical outcomes may be patient reported. The clinical outcomes may be reported by a clinician. The clinical outcomes may be reported by an observer. The clinical outcomes may comprise measurements, e.g. blood glucose measurements. The user information may include data concerning one or more performances of the user, e.g. results of a hand tremor status.

The user information of each first record may be freely defined by the user, optionally comprising one or more choices made from respective groups of predetermined alternatives.

The user may be allowed to record input such as observations when preferred by the user. The method may enable time stamping the user information and verifying reliability of the time stamping with and without network access.

The method may further comprise verifying reliability of the recorded current clock times of the first entries. The verifying may be performed using the second entries.

The method may enable verifying the reliability of the recorded current clock times solely based on the information available to the portable digital device without access to the server. Additionally, the method may enable verifying the reliability of the recorded current clock times using the information available to the server.

The verifying may be performed by the server. Alternatively or additionally, the verifying may be performed by the portable digital device.

The recording of the first and second entries may be performed by a time tracking module of the portable digital device. The communicating with the server may be performed by the time tracking module. The obtaining of the current reference time from the server may be performed by the time tracking module. The using of the encryption may be performed by the time tracking module.

The second entries may be recorded in the portable digital device. Alternatively, the second entries may be recorded to the server by providing the current clock time and uptime. The server may record the reference time on recording the second entry. The server may provide the portable digital device with the reference time.

The method may enable maintaining reference time records by the server for comparison with the first entries recorded at the portable digital device.

The method may further comprise maintaining a boot number counter by the portable digital device or by the time tracking module. The method may further comprise continually checking if the boot number counter value has increased and if yes, responsively performing the reference time recording process. The boot number counter value may be additionally recorded to next first entry. The boot number counter value may be additionally recorded to the second entry on performing the reference time recording process responsive to the increasing of the boot number counter value.

The method may further comprise determining whether the boot number counter value has increased by more than one since previously recorded combination of boot number counter value, uptime and current clock time. If yes, the method may comprise recording a first indication to a subsequently recorded first entry. The first indication may indicate unreliability of the clock time until next performing of the reference time recording process.

The use of the boot number counter may enable checking whether it is still possible to track backwards the correct time for each recorded entry based on the uptime.

The method may further comprise determining whether the portable digital device has been restarted once after recording both the previous first and second entries and if yes, recording next first entry with a third indication. The third indication may indicate one restart since recording previous first and second entries.

The method may comprise determining, from the recorded current uptime and the reference time of the second entries, if the portable digital device has been restarted and if yes, subtracting the uptime from the current clock time and recording the result as previous starting time.

The method may further comprise determining whether the previous starting time calculated for one first entry is later than that calculated for a subsequent first entry and if yes, determining that at least one of these first entries is erroneous. The method may further comprise determining, based on the reference time whether the subsequent first entry had a correct clock time and if yes, then determining the one first entry and the subsequent first entry respectively as erroneously and correctly timed.

The method may enable determining the presence of impossible sequences of time stamps as indication of errors in the time stamping.

The method may comprise compensating time zone changes by detecting abrupt changes in the difference between the recorded current clock time and either the uptime or reference time, when the difference is N times half an hour, wherein N is an integer between −47 and −1 or +1 and +47.

The verifying may comprise determining, for one uptime period, a difference between the recorded current uptime and the current clock time of the second entries and checking whether that difference has changed more than a predetermined first maximum change. The uptime period may refer to the period from starting up (e.g., cold boot, warm boot, power on) until a next shutdown or restart of the portable digital device. The first maximum change may be greater than 10 s, 30 s, 1 min, 2 min, 5 min or 10 min. The first maximum change may be per 24 hours greater than 10 s, 30 s, 1 min, 2 min, 5 min or 10 min.

The verifying may comprise determining, for one uptime period, a difference between the recorded current uptime and the current clock time of the first entries and checking whether that difference has changed more than a predetermined second maximum change. The second maximum change may be greater than 10 s, 30 s, 1 min, 2 min, 5 min or 10 min

The method may comprise determining which ones if any of the first entries the recorded current uptime and current clock time mutually change more than by second maximum change and recording to such first entries a second indication. The second indication may indicate unreliable current clock time.

The using encryption by the portable digital device may comprise encrypting the first entries. The using encryption by the portable digital device may comprise encrypting the second entries. The using encryption by the portable digital device may comprise cryptographically signing the first entries. The using encryption by the portable digital device may comprise cryptographically signing the second entries. The encryption may employ asymmetric encryption. The encryption may employ public key encryption. The encryption may employ symmetric encryption.

The second entry may comprise user information. The second entry may be one of the first entries. The recording of the user information in some second entries or having the second entry as one of the first entries may remove the need of recording separate first and second entries.

The second entry may be recorded one or more times before first time of recording the first entry.

According to a second example aspect of the invention there is provided a portable digital device comprising:
at least one circuitry configured to maintain a current clock time and a current uptime;
at least one processor configured to:
   record by the portable digital device a plurality of times a first entry comprising:
      user information;
      the current clock time;
      the current uptime;
      perform one or more times a reference time recording process comprising recording by the portable digital device together with a server, which server has a reference time, a second entry that comprises:

The at least one processor may be further configured to cause providing the server with the first and second entries.

The at least one processor may be further configured to use encryption such that access to content of the first and second entries is inhibited and integrity of the first and second entries is protected.

According to a third example aspect of the invention there is provided a computer program comprising computer executable program code which when executed by at least one processor causes an apparatus at least to perform the method of the first example aspect.

The computer program may be protected against reverse engineering. The computer program may be code obfuscated. The computer program may be configured to decrypt and sandbox the computer program code for run time.

According to a fourth example aspect of the invention there is provided a computer program product comprising a non-transitory computer readable medium having the computer program of the third example aspect stored thereon.

Any foregoing memory medium may comprise a digital data storage such as a data disc or diskette, optical storage, magnetic storage, holographic storage, opto-magnetic storage, phase-change memory, resistive random access memory, magnetic random access memory, solid-electrolyte memory, ferroelectric random access memory, organic memory or polymer memory. The memory medium may be formed into a device without other substantial functions than storing memory or it may be formed as part of a device with other functions, including but not limited to a memory of a computer, a chip set, and a sub assembly of a digital device.

Different non-binding example aspects and embodiments of the present invention have been illustrated in the foregoing. The embodiments in the foregoing are used merely to explain selected aspects or steps that may be utilized in implementations of the present invention. Some embodiments may be presented only with reference to certain example aspects of the invention. It should be appreciated that corresponding embodiments may apply to other example aspects as well.

### BRIEF DESCRIPTION OF THE DRAWINGS

Some example embodiments of the invention will be described with reference to the accompanying drawings, in which:

FIG. 1 shows a schematic drawing of a system of an embodiment;

FIG. 2 shows a block diagram of a portable digital device of an embodiment;

FIG. 3 shows a block diagram of a server of an embodiment of the invention; and

FIGS. 4a to 4c show a flow chart of an embodiment.

### DETAILED DESCRIPTION

In the following description, like reference signs denote like elements or steps.

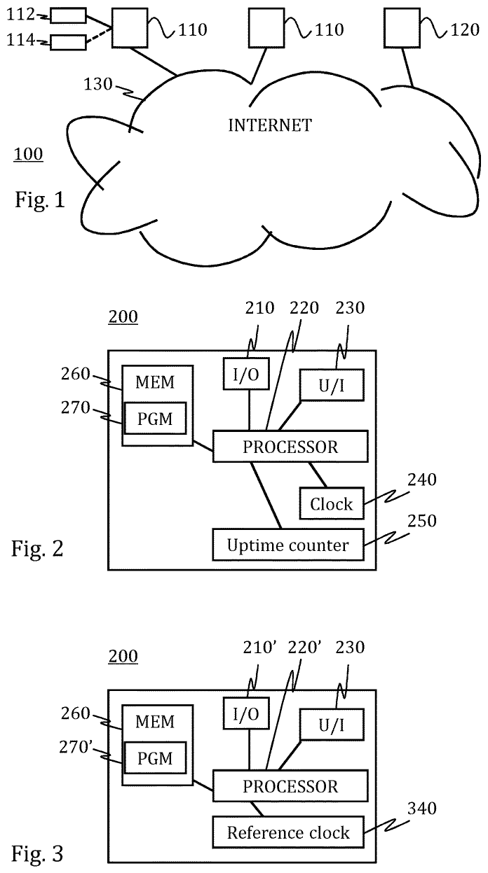

FIG. 1 shows a schematic drawing of a system 100 of an embodiment. The system 100 comprises a plurality of portable digital devices 200 and a server 120 connected to the Internet 130. In an embodiment, the system further comprises one or more wired measuring devices 112 and/or wireless measuring devices 114. The measuring devices 112, 114 may be configured to communicate with the portable digital device 200. The wired or wireless measuring devices may comprise any one or more of: a glucometer; a continuous glucometer; a pulse sensor; a pedometer; a thermometer; an electrocardiography sensor.

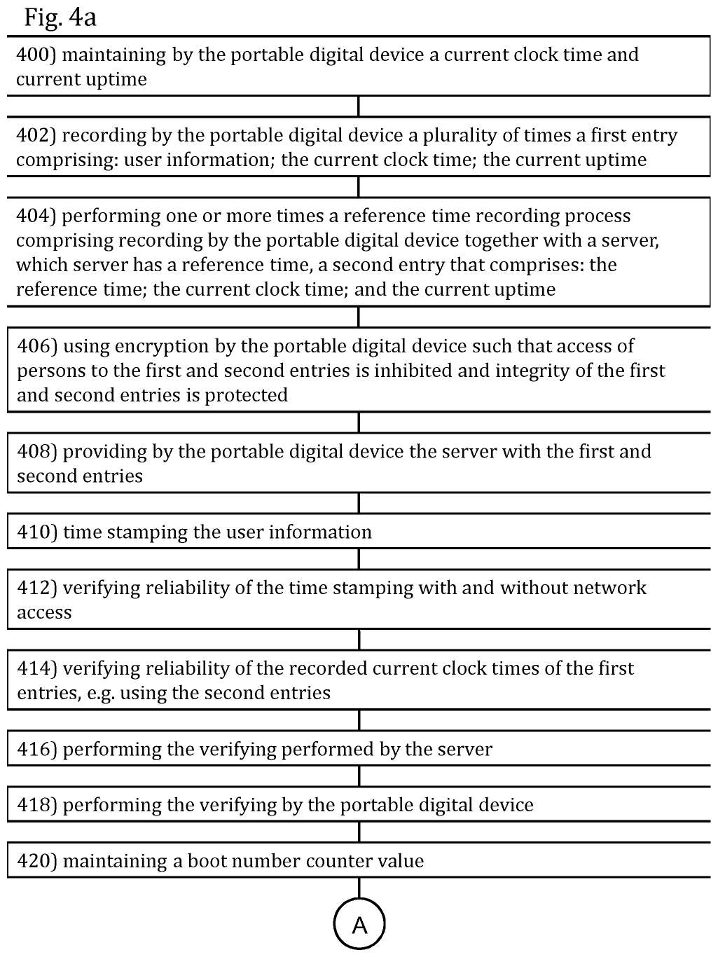

FIG. 2 presents an example block diagram of a portable digital device 200 in which various embodiments of the invention may be applied. This may be a user device or apparatus, such as a cellular telephone, tablet computer, laptop computer, electric book, portable gaming device, or a smart watch.

The general structure of the portable digital device 200 comprises a communication interface 210, a processor 220, and a memory 260. The portable digital device further comprises software 270 stored in the memory 260 and operable to be loaded into and executed in the processor 220. The software 270 may comprise one or more software modules and can be in the form of a computer program product. The portable digital device 200 further comprises a user interface 230. Still further there are a clock 240 for maintaining current clock time and an uptime counter 250 for maintaining the uptime. Notice that the separately drawn entities need not be discrete or separate from each other, they are just drawn this way in sake of simplicity.

The communication interface 210 comprises, for example, a base 100 or base 1000 Ethernet connection; a local bus; a Bluetooth circuitry; a short range radio communication circuitry; an ANT circuitry; an ANT+ circuitry; a universal serial bus circuitry; a parallel and/or a serial link circuitry. The communication interface 210 is configured in an example embodiment to communicate with one circuitry with the Internet and with the same or one or more other circuitries with the wired and/or wireless measuring devices 112, 114.

The processor 220 is, or comprises, e.g., a central processing unit (CPU), a microprocessor, a digital signal processor (DSP), a graphics processing unit, an application specific integrated circuit (ASIC), a field programmable gate array, a microcontroller or a combination of such elements. FIG. 2 shows one processor 220, but the portable digital device 200 may comprise a plurality of processors.

The memory 260 may be for example a volatile or a non-volatile memory, such as a read-only memory (ROM), a programmable read-only memory (PROM), erasable programmable read-only memory (EPROM), a random-access memory (RAM), a flash memory, a data disk, an optical storage, a magnetic storage, a smart card, or the like. The portable digital device 200 may comprise a plurality of memories. The memory 260 can be constructed as a part of the portable digital device 200 or it may be inserted into a slot, port, or the like of the portable digital device 200 by a user. The memory 260 may serve the sole purpose of storing data, or it may be constructed as a part of an apparatus serving other purposes, such as processing data.

The user interface 230 comprises, for example, a circuitry for receiving input from a user of the portable digital device 200, e.g., via a keyboard, graphical user interface shown on the display of the portable digital device 200, speech recognition circuitry, or an accessory device, such as a headset, and for providing output to the user via, e.g., a graphical user interface or a loudspeaker.

A skilled person appreciates that in addition to the elements shown in FIG. 2, the portable digital device 200 may comprise other elements, such as microphones, displays, as well as additional circuitry such as input/output (I/O) circuitry, memory chips, application-specific integrated circuits (ASIC), processing circuitry for specific purposes such as source coding/decoding circuitry, channel coding/decoding circuitry, ciphering/deciphering circuitry, and the like. Additionally, the portable digital device 200 may comprise a disposable or rechargeable battery (not shown) for powering the portable digital device 200 when external power if external power supply is not available.

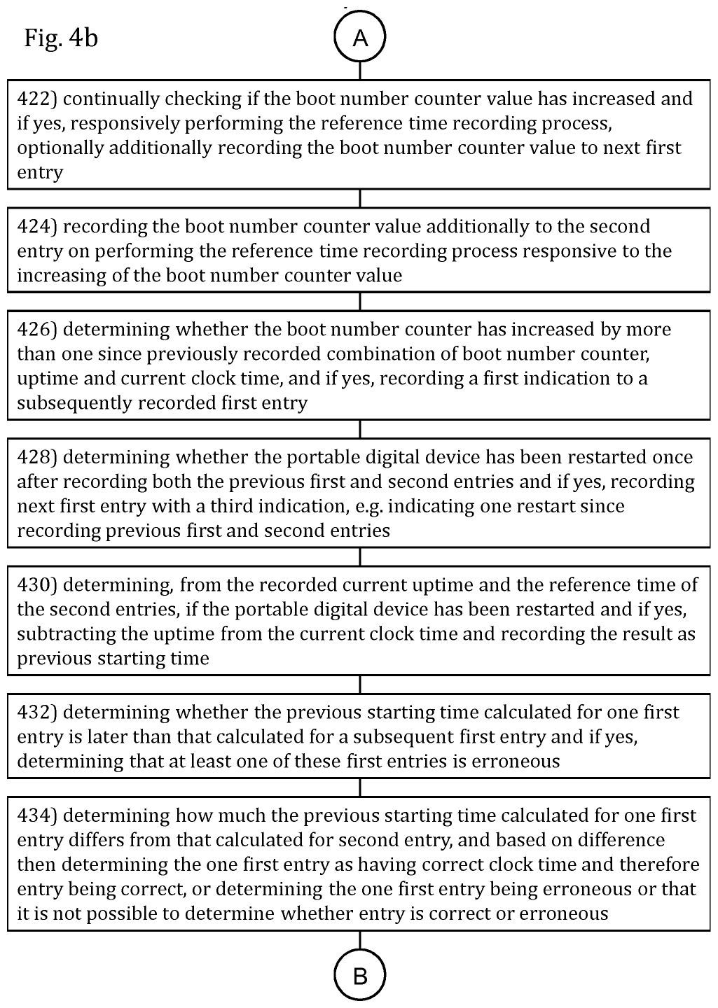

FIG. 3 shows a block diagram of a server 120 of an embodiment of the invention. The general structure of the server 300 comprises a communication interface 210′, a processor 220′, and a memory 260. The server 300 further comprises software 270′ stored in the memory 260 and operable to be loaded into and executed in the processor 220′. The software 270′ may comprise one or more software modules and can be in the form of a computer program product. The server 300 further comprises the user interface 230. Still further there is a reference clock 340 for maintaining current clock time. Notice that the separately drawn entities need not be discrete or separate from each other, they are just drawn this way in sake of simplicity.

The communication interface 210′ comprises, for example, a base 100 or base 1000 Ethernet connection, a local bus, a parallel and/or a serial link circuitry.

The processor 220′ is, or comprises, e.g., a central processing unit (CPU), a microprocessor, a digital signal processor (DSP), a graphics processing unit, an application specific integrated circuit (ASIC), a field programmable gate array, a microcontroller or a combination of such elements. FIG. 2 shows one processor 220′, but the server 300 may comprise a plurality of processors.

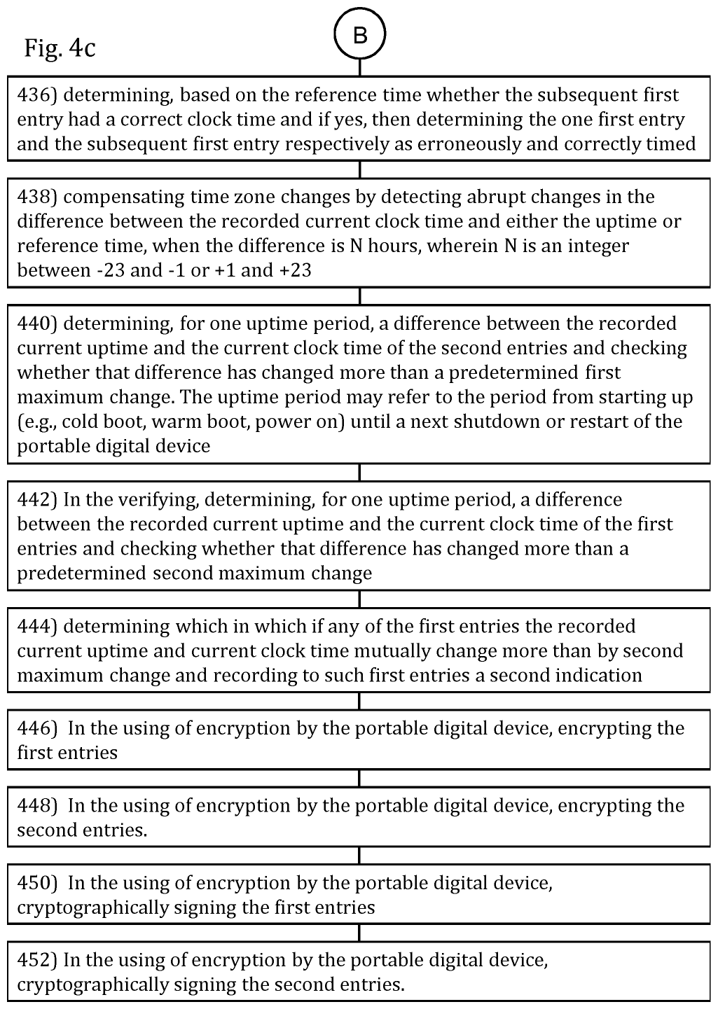

FIGS. 4a to 4c show a flow chart of an embodiment, illustrating an automatic time tracking and validating method of a portable digital device, comprising:
400) maintaining by the portable digital device a current clock time and current uptime;
402) recording by the portable digital device a plurality of times a first entry comprising: user information; the current clock time; the current uptime;
404) performing one or more times a reference time recording process comprising recording by the portable digital device together with a server, which server has a reference time, a second entry that comprises: the reference time; the current clock time; and the current uptime;
406) using encryption by the portable digital device such that access of persons to the first and second entries is inhibited and integrity of the first and second entries is protected;
408) providing by the portable digital device the server with the first and second entries.

In an embodiment, the recording of the first entry is performed in response to the portable digital device receiving from a user the user information. The receiving of the user information can be started on initiative of the user. Alternatively, or additionally, the portable digital device 200 can be configured to prompt the user for the user information at given times. The user information of each first record can be freely defined by the user, optionally comprising one or more choices made from respective groups of predetermined alternatives. Further alternatively or additionally, the user information may comprise automatically obtained information such as measurement information received from the wired or wireless measurement device 112, 114.

The method may further comprise any one or more of:
410) time stamping the user information;
412) verifying reliability of the time stamping with and without network access.
414) verifying reliability of the recorded current clock times of the first entries, e.g. using the second entries;
416) performing the verifying performed by the server;
418) performing the verifying by the portable digital device.

In an embodiment, the recording of the first and second entries is performed by a time tracking module of the portable digital device. The communicating with the server can be performed by the time tracking module. The obtaining of the current reference time from the server can be performed by the time tracking module. The using of the encryption can be performed by the time tracking module.

In an embodiment, the second entries are recorded in the portable digital device. Alternatively, the second entries may be recorded to the server by providing the current clock time and uptime. The server may record the reference time on recording the second entry. The server may provide the portable digital device with the reference time.

In an embodiment, the method further comprises any one or more of:
420) maintaining a boot number counter. The boot number counter may be maintained by the portable digital device. In an embodiment, the boot number counter is maintained by the time tracking module.
422) continually (in some embodiments periodically, in some other embodiments irregularly or with changing periods of time) checking if the boot number counter value has increased and if yes, responsively performing the reference time recording process. The boot number counter value may be additionally recorded to next first entry.
424) recording the boot number counter value additionally to the second entry on performing the reference time recording process responsive to the increasing of the boot number counter value.
426) determining whether the boot number counter value has increased by more than one since previously recorded combination of boot number counter value, uptime and current clock time, and if yes, recording a first indication to a subsequently recorded first entry;
428) determining whether the portable digital device has been restarted once after recording both the previous first and second entries and if yes, recording next first entry with a third indication, e.g. indicating one restart since recording previous first and second entries;
430) determining, from the recorded current uptime and the reference time of the second entries, if the portable digital device has been restarted and if yes, subtracting the uptime from the current clock time and recording the result as previous starting time.
432) determining whether the previous starting time calculated for one first entry is later than that calculated for a subsequent first entry and if yes, determining that at least one of these first entries is erroneous;
434) determining how much the previous starting time calculated for one first entry differs from that calculated for second entry, and based on difference then determining the one first entry as having correct clock time and therefore entry being correct, or determining the one first entry being erroneous or that it is not possible to determine whether entry is correct or erroneous;
436) determining, based on the reference time whether the subsequent first entry had a correct clock time and if yes, then determining the one first entry and the subsequent first entry respectively as erroneously and correctly timed.
438) compensating time zone changes by detecting abrupt changes in the difference between the recorded current clock time and either the uptime or reference time, when the difference is N hours, wherein N is an integer between −23 and −1 or +1 and +23;
440) determining, for one uptime period, a difference between the recorded current uptime and the current clock time of the second entries and checking whether that difference has changed more than a predetermined first maximum change. The uptime period may refer to the period from starting up (e.g., cold boot, warm boot, power on) until a next shutdown or restart of the portable digital device. The first maximum change may be greater than 10 s, 30 s, 1 min, 2 min, 5 min or 10 min. The first maximum change may be per 24 hours greater than 10 s, 30 s, 1 min, 2 min, 5 min or 10 min.
442) In the verifying, determining, for one uptime period, a difference between the recorded current uptime and the current clock time of the first entries and checking whether that difference has changed more than a predetermined second maximum change;
444) determining which in which if any of the first entries the recorded current uptime and current clock time mutually change more than by second maximum change and recording to such first entries a second indication. The second indication may indicate unreliable current clock time.
446) In the using of encryption by the portable digital device, encrypting the first entries.
448) In the using of encryption by the portable digital device, encrypting the second entries.
450) In the using of encryption by the portable digital device, cryptographically signing the first entries.
452) In the using of encryption by the portable digital device, cryptographically signing the second entries.

In an embodiment, the encryption employs public key encryption.

In an embodiment, the second entry comprises user information. In an embodiment, the second entry is one of the first entries. The recording of the user information in some second entries or having the second entry as one of the first entries may remove the need of recording separate first and second entries.

Various embodiments have been presented. It should be appreciated that in this document, words comprise, include and contain are each used as open-ended expressions with no intended exclusivity.

The foregoing description has provided by way of non-limiting examples of particular implementations and embodiments of the invention a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed embodiments of this invention may be used to advantage without the corresponding use of other features. As such, the foregoing description shall be considered as merely illustrative of the principles of the present invention, and not in limitation thereof. Hence, the scope of the invention is only restricted by the appended patent claims.

1. An automatic time tracking and validating method of a portable digital device, comprising:
maintaining by the portable digital device a current clock time and current uptime;
recording by the portable digital device a plurality of times a first entry comprising:
user information;
the current clock time; the current uptime;
performing one or more times a reference time recording process comprising:
recording by the portable digital device together with a server, which server has a reference time, a second entry that comprises: the reference time; the current clock time; and the current uptime;
the method further comprising providing by the portable digital device the server with the first and second entries.

2. The method of claim 1, wherein the recording of the first entry is performed in response to the portable digital device receiving from a user the user information.

3. The method of claim 1, wherein the user information comprises information provided by the user.

4. The method of claim 1, wherein the method further comprises verifying reliability of the recorded current clock times of the first entries.

5. The method of claim 1, further comprising using encryption by the portable digital device such that access to the first and second entries is inhibited and integrity of the first and second entries is protected.

6. The method of claim 1, wherein the user information is measured from the user.

7. The method of claim 1, wherein the user information of each first record is defined by the user.

8. The method of claim 1, wherein the user is allowed to record input when preferred by the user.

9. The method of claim 8, wherein the verifying is performed using the second entries.

10. The method of claim 1, wherein the verifying is performed by the server.

11. The method of claim 1, wherein the recording of the first and second entries is performed by a time tracking module of the portable digital device.

12. The method claim 11, wherein the obtaining of the current reference time from the server is performed by the time tracking module.

13. The method of claim 1, wherein the second entries are recorded in the portable digital device.

14. The method of claim 1, wherein the server provides the portable digital device with the reference time.

15. The method of claim 1, further comprising maintaining a boot number counter by the portable digital device or by the time tracking module.

16. The method of claim 15, further comprising continually checking if the boot number counter value has increased and if yes, responsively performing the reference time recording process.

17. The method of claim 16, wherein the boot number counter value is additionally recorded to next first entry.

18. The method of claim 17, wherein the boot number counter value is additionally recorded to the second entry on performing the reference time recording process responsive to the increasing of the boot number counter value.

19. The method of claim 18, further comprising determining whether the boot number counter value has increased by more than one since previously recorded combination of boot number counter value, uptime and current clock time.

20. The method of claim 19, further comprising, if the boot number counter value has increased by more than one since previously recorded combination of boot number counter value, uptime and current clock time, performing recording a first indication to a subsequently recorded first entry.

21. The method of claim 20, further wherein the first indication indicates unreliability of the clock time until next performing of the reference time recording process.

22. The method of claim 1, further comprising determining, from the recorded current uptime and the reference time of the second entries, if the portable digital device has been restarted and if yes, subtracting the uptime from the current clock time and recording the result as previous starting time.

23. The method of claim 1, further comprising determining whether the previous starting time calculated for one first entry is later than that calculated for a subsequent first entry and if yes, determining that at least one of these first entries is erroneous.

24. The method of claim 1, further comprising determining how much the previous starting time calculated for one first entry differs from that calculated for second entry, and based on difference then determining the one first entry as having correct clock time and therefore entry being correct, or determining the one first entry being erroneous or that it is not possible to determine whether entry is correct or erroneous.

25. The method of claim 1, further comprising determining, based on the reference time whether the subsequent first entry had a correct clock time and if yes, then determining the one first entry and the subsequent first entry respectively as erroneously and correctly timed.

26. The method of claim 1, further comprising compensating time zone changes by detecting abrupt changes in the difference between the recorded current clock time and either the uptime or reference time, when the difference is N times half an hour, wherein N is an integer.

27. The method of claim 1, wherein the verifying comprises determining, for one uptime period, a difference between the recorded current uptime and the current clock time of the second entries and checking whether that difference has changed more than a predetermined first maximum change.

28. The method of claim 1, wherein the verifying comprises determining, for one uptime period, a difference between the recorded current uptime and the current clock time of the first entries and checking whether that difference has changed more than a predetermined second maximum change.

29. The method of claim 1, wherein the method comprises determining which ones if any of the first entries the recorded current uptime and current clock time mutually change more than by second maximum change and recording to such first entries a second indication.

30. The method of claim 1 The method of any one of preceding claims, further comprising determining whether the portable digital device has been restarted once after recording both the previous first and second entries and if yes, recording next first entry with a third indication.

31. The method of claim 1, wherein the second entry comprises user information.

32. The method of claim 1, wherein the second entry is one of the first entries.

33. A portable digital device comprising:
at least one circuitry configured to maintain a current clock time and a current uptime;
at least one processor configured to perform with the at least one circuitry at least the method of claim 1.

34. A computer program comprising computer executable program code which when executed by at least one processor causes an apparatus at least to perform the method of claim 32.

35. A computer program product comprising a non-transitory computer readable medium having the computer program of claim 34.
